(19) United States
(12) Patent Application Publication
(10) Pub. No.: US 2021/0010066 A1
(43) Pub. Date: Jan. 14, 2021

(54) DEVICES AND METHODS FOR NUCLEIC ACID IDENTIFICATION

(71) Applicant: Niagara Biosciences, Inc., Arlington, MA (US)

(72) Inventor: Vishal A. Patil, Arlington, MA (US)

(73) Assignee: Niagara Biosciences, Inc., Arlington, MA (US)

(21) Appl. No.: 16/922,876

(22) Filed: Jul. 7, 2020

Related U.S. Application Data
(60) Provisional application No. 62/873,671, filed on Jul. 12, 2019.

(51) Int. Cl. C12Q 1/689; G16B 15/10; G01N 21/64

(52) U.S. Cl. CPC C12Q 1/689; G01N 2021/6439; G01N 21/6428; G16B 15/10

(57) ABSTRACT

Discussed herein are devices and methods for obtaining nucleotide sequence information from nucleic acid and nucleic acid samples. The device includes a fluidic channel that aids in manipulating a sample as the sample flows through various zones of the channel. Provided herein are methods for nucleic acid analysis that help to identify and filter out molecules that are in non-ideal conformations such as being folded, kinked, or overlapping with other molecules.

### CROSS REFERENCE TO RELATED APPLICATIONS

This Application claims the benefit under 35 U.S.C. § 119(e) of U.S. Provisional Application Ser. No. 62/873,671, entitled “DEVICES AND METHODS FOR NUCLEIC ACID IDENTIFICATION” filed on Jul. 12, 2019, the entire contents of which is incorporated herein by reference.

### BACKGROUND

Bacterial typing/identification can be achieved by mapping bacteria's DNA. DNA mapping can give information about a location and relative distance between a gene on a body of DNA. Single molecule DNA mapping may involve digestion of linearized DNA by restriction enzymes (RE) due to RE's selectivity for their cognate sites. Some approaches for linearizing DNA molecules use nanochannels or combing.

DNA combing involves stretching and fixing DNA molecules, via electrostatic interactions, onto a glass surface. In nanochannel-based devices, intercalated DNA is stretched by confinement. To work effectively, both techniques require appropriate RE selection. Moreover, both the techniques involve DNA-wall interaction, which may require additional sample purification processes. Long DNA sample prep can be nontrivial because of the limitations on the use of mechanical forces. Real environmental samples are likely to have microbes that cannot be completely lysed, and an overall DNA purity that is lower than that of cultured samples.

### SUMMARY

The stochastic process of nucleic acid molecule stretching produces various conformational states. For higher detection efficiency and sensitivity, most of the nucleic acid molecules entering the cutting zone of a fluidic device need to be well stretched and non-overlapping so that the RE sites are linearized and digested trains are not intermingled. Possible non-ideal scenarios include, but are not limited to, molecules overlapping with one another, and a molecule that is folded onto itself. Data analysis methods which utilize spacing between fluorescent signal peaks to detect the non-ideal scenarios are discussed herein.

The devices and methods described herein can be used with virtually any nucleic acid (e.g., DNA) sample, and requires minimal manipulation of the sample.

The methods provided herein may be performed on single nucleic acids such as single DNA molecules, and are thus referred to as “single molecule analysis” methods. The nucleic acids are typically not fixed or immobile (e.g., conjugated to a support such as a bead or a surface) and rather are in flow in a fluid stream.

One aspect of this disclosure provides a method in relation to identifying a nucleic acid of a sample, the method being for determining whether a molecule of the sample exhibits a predetermined physical characteristic. The method includes an act of: (A) determining a distance g between a first fragment and a second fragment of the sample, based at least in part upon an amount of time G between a first signal vector element P1 representing emission of light by the first fragment and a second signal vector element P2 representing emission of light by the second fragment, the first signal vector element P1 and second signal vector element P2 contributing to a sum Psum of all elements of signal vectors representing emission of light by a plurality of fragments including the first fragment and the second fragment. The method also includes acts of: (B) determining at least one of a distance threshold and a signal vector threshold; (C) performing a comparison between the distance and the distance threshold, between the sum of all the elements of signal vectors and the signal vector threshold, or between the distance and the distance threshold and between the sum of all the elements of signal vectors and the signal vector threshold; and (D) determining based at least in part upon the comparison performed in the act (C) whether the molecule exhibits the predetermined physical characteristic.

These and other aspects and embodiments will be described in greater detail herein.

### BRIEF DESCRIPTION OF DRAWINGS

FIG. 1 is a schematic illustrating a free solution genome mapping method of analyzing nucleic acids according to one aspect. While the figure refers to staining of DNA with PicoGreen®, it is to be understood that other intercalators can be used, including for example SYBER™ Green. Intercalators that do not significantly impact DNA structure upon binding to DNA may be used in such embodiments. Such intercalators are known in the art.

FIG. 2 is an embodiment of a device that may be used to implement the method of FIG. 1.

FIG. 3 is an illustration of a situation where DNA size, 1_{DNA}, in direction of flow is smaller than an interrogation region's size, 1_{IR}.

FIG. 4 is an illustration of a case where DNA size, 1_{DNA}, in direction of flow is larger than an interrogation region's size, 1_{IR}.

FIG. 5 is an illustration of a train of intercalated digested DNA fragments passing through an interrogation region.

FIG. 6 is a graph of a fluorescent signal that is collected at discrete intervals and is curve fitted to provide peak intensity vector (in photon counts) for a single DNA molecule entering the fluidic device.

FIG. 7 depicts the device of FIG. 2 with velocity vector fields and the state of a nucleic acid flowing through the device illustrated at various zones of the device.

FIG. 8 depicts various possible conformational states of DNA molecules entering the cutting zone of the fluidic device.

FIG. 9A is a graph of a fluorescent signal that is collected at discrete intervals and is curve fitted to provide a peak spacing vector (in seconds).

FIG. 9B depicts a train of intercalated digested nucleic acid fragments with spacing g in meters passing through the interrogation region.

FIG. 10 depicts a DNA fragment modeled as a soft particle in flow.

FIG. 11A depicts a DNA molecule in a stretched state with REs occupying cognate sites.

FIG. 11B depicts a hypothetical model representing a DNA-RE complex as chain of soft particles connected at restriction sites.

FIG. 11C depicts particles becoming independent of each other when the REs complete hydrolysis.

FIG. 12 depicts behavior of two sets of particles released at different positions in converging flow.

FIG. 13 depicts behavior of two sets of particles released at different orientations in converging flow.

FIG. 14 depicts behavior of two sets of particles, one set being of equal size and the other set being of unequal size with larger average particle diameter, exhibiting different separation due to flow.

FIG. 15 depicts an ideal case in which the acceleration flow field in a focusing zone of a fluidic device results in nucleic molecule stretching and separation before entry into a cutting zone of a fluidic device.

FIG. 16 depicts a case in which overlapped nucleic acid molecules have entered the cutting zone.

FIG. 17 depicts a case in which folded nucleic acid molecules have entered the cutting zone.

FIG. 18 depicts a histogram of normalized gap gₙₒᵣₘ between fragments for setting an upper threshold value g_{thres,high}.

FIG. 19 depicts a histogram of a sum of elements of a peak intensity vector Pₛᵤₘ for determining lower and upper thresholds P_{sum,low} and P_{sum,high}, respectively, for filtering out peak spacing and peak intensity vectors from nucleic acid molecules which are overlapped, or are too large for complete stretching in the fluidic device, or have undergone tension induced breaks.

FIG. 20 depicts a histogram of normalized gap between fragments from individual nucleic acid molecules for setting a lower threshold value g_{thres,low}.

### DETAILED DESCRIPTION

One type of approach to obtaining the fingerprint of a nucleic acid is described in International Publication No. WO 2018/106897, filed Dec. 7, 2017, the disclosure of which is incorporated by reference herein. The approach generally includes use of a fluidic device having various different zones, each having a designated purpose. The approach generally includes linearizing/stretching nucleic acid molecules in flow, cutting the linearized/stretched nucleic acid molecules using restriction enzymes (REs), then passing the digested fragments through a relaxation zone that encourages coil formation of the digested fragments and introduces gaps between the digested fragments. The fragments then pass through a detection zone. In some embodiments, the nucleic acid molecules are stained with a fluorescent marker prior to introducing the molecules into the analysis device, and fluorescent intensity is measured at the detection zone to detect passage of a digested fragment through the detection zone.

The inventor has appreciated that nucleic acid fragments passing through the detection zone can take on various conformations, including non-ideal conformations that may interfere with analysis. The inventor has appreciated that, for example, if fragments are not well separated, or if some nucleic acid molecules were not well-stretched, such situations may give rise to inaccurate measurement results. The inventor has recognized the need for methods to detect when molecules having non-ideal conformations are passing through the detection zone in order to exclude the measurements associated with these molecules. Provided herein are methods for nucleic acid analysis that help to identify and filter out molecules that are in non-ideal conformations, such as not well stretched and/or not separated from other molecules.

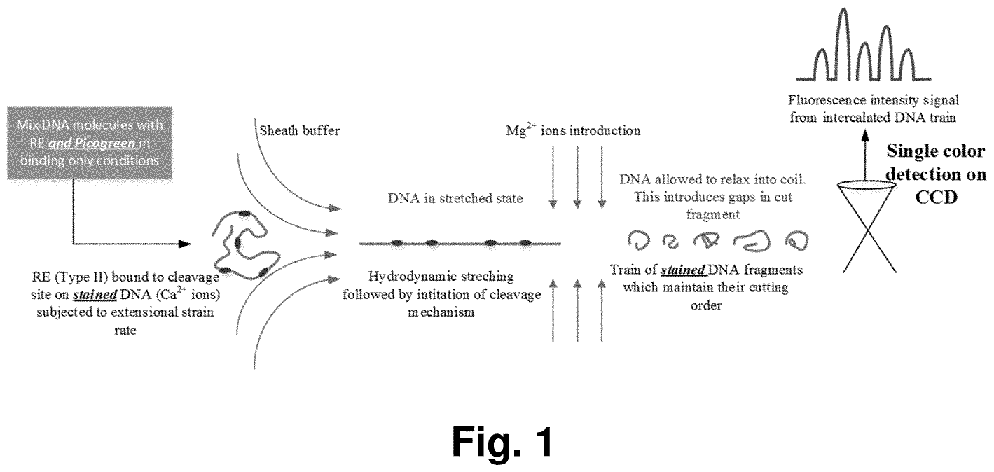

One embodiment of an approach for analyzing nucleic acids is provided in FIG. 1, which illustrates the order of operations to be performed for mapping a single nucleic acid (e.g., DNA) in a fluidic device. In some embodiments, the order of operation is DNA stretching, Mg2+ ion (or other suitable agent or condition) introduction, cutting of DNA (also referred to here as hydrolysis of DNA), gap formation between cut fragments, and optical detection.

It is to be understood that although various descriptions provided herein may refer to DNA, these descriptions apply more broadly to nucleic acids in general unless explicitly stated otherwise.

As used herein, a nucleic acid in flow means a nucleic acid that is not attached at any point to a solid support. Thus, the nucleic acid moves along in a sheath fluid while the various manipulations and modifications described herein are performed.

FIG. 1 illustrates the various steps starting from mixing the harvested DNA with RE, such as a type II RE, and an intercalator, such as PicoGreen®, in a binding condition (e.g., Ca2+, no Mg2+, no Mn2+), hydrodynamic stretching of the DNA and exposure of the DNA to the cleaving condition (e.g., introduction of Mg2+ ions), allowing the DNA to relax into a coiled state, optionally increasing the distance between digested fragments, detecting the fragments using, for example, a single color detector such as APD, PMT, CCD and CMOS, data collection and analysis. Subsequent steps such as creation of a map or signature of the parent DNA (based on the proportional relationship between fluorescence intensity and DNA length), pattern assembly/matching (e.g., arranging various fragments relating to each other, including with overlap between fragments), and, optionally, identification of one or more microbes in the original complex sample, are also contemplated. The DNA may be labeled with an intercalator before and/or during and/or after incubation with RE, including optionally under the binding conditions, without significant (if any) impact on RE binding.

In some embodiments, the device used to implement the approach of FIG. 1 is a microfluidic device and may have a microfluidic channel having a sample inlet, a focusing zone having a geometry that aids in stretching of nucleic acids flowing through the focusing zone, a cutting zone having geometry to keep the nucleic acid in a stretched state during digestion by RE, a relaxation zone having a geometry that aids in relaxation of nucleic acids, and a detection zone. In some embodiments, the device may have one or more supplementary inlets that allow the addition of agents or other fluids into the channel.

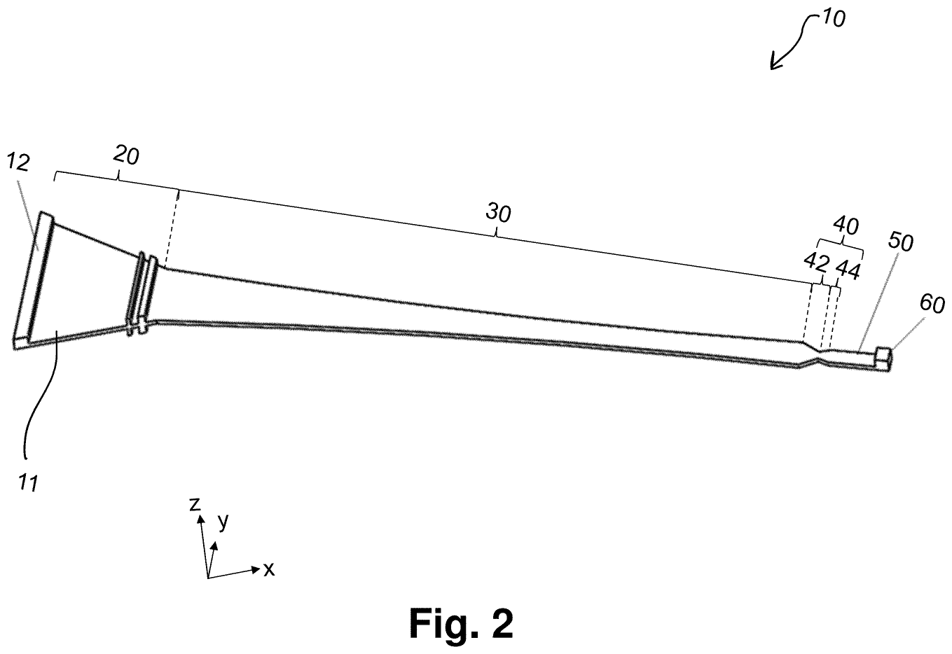

One illustrative embodiment of a fluidic device is provided in FIG. 2. The device may be used for nucleic acid mapping in free solution. The device 10 comprises a channel 11 through which a sample is flowed. The channel 11 has an inlet 12 for receiving the sample, and may include an outlet 60 through which sample is discharged. The channel may be a microfluidic channel. In some embodiments, the channel may have a plurality of zones, including: a focusing zone 20, cutting zone 30, relaxation zone 40, and detection zone 50.

While a high level description of the features and functions of each zone of the fluidic device is provided below, a more detailed description can be found in International

Publication No. WO 2018/106897, filed Dec. 7, 2017, which, as stated above, the disclosure of which is incorporated by reference herein.

The focusing zone may be used for one or a combination of the following functions: stretching the nucleic acids, introducing cutting agents, introducing viscosifying agents, and/or reducing sample depth for mixing and optical focusing. In some embodiments, the focusing zone may have a converging width shape and/or may be preceded by a converging width shape section to aid nucleic acid stretching. In the embodiment shown in FIG. 2, the focusing zone 20 has a converging width shape.

The cutting zone, in some embodiments, may serve to keep nucleic acids and their fragments stretched until the hydrolysis reaction by the REs bound to the nucleic acid is complete. The cutting zone may help to provide tension to keep the nucleic acids stretched. In some embodiments, the cutting zone of the channel has a converging width shape to hydrodynamically provide tension on the nucleic acids. As shown in FIG. 2, the cutting zone 30 of the channel has a converging width shape.

The relaxation zone, in some embodiments, may serve to introduce gaps in the digested nucleic acid fragments in order to help distinguish one fragment from another during detection. In this zone, the once-stretched nucleic acids are permitted to relax and consequently coil back. In some embodiments, to promote nucleic acid coiling, the relaxation zone includes a flow acceleration zone followed by a deceleration zone. In some embodiments, such as the embodiment shown in FIG. 2, the relaxation zone 40 has an acceleration zone 42 with a converging width shape and a deceleration zone 44 with a diverging width shape.

In the detection zone, the nucleic acid fragments are detected and identified. In some embodiments, the detection zone may be part of the channel. In some embodiments, such as in the illustrative embodiment of FIG. 2, the channel walls of the detection zone 50 may be straight.

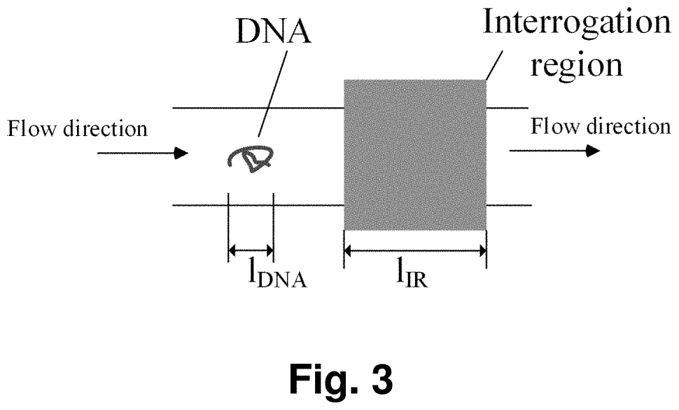

In some embodiments, fluorescent intensity from stained nucleic acids is measured to estimate size of the nucleic acid. As illustrated in FIG. 3, the detection zone may have an interrogation region in which the fluorescent intensity emitted by excited stained DNA fragments would be collected and focused onto a detector such as avalanche photodiodes (APDs) or photomultiplier tubes (PMTs). If physical DNA size, 1_{DNA} is smaller than an interrogation region size, 1_{IR}, then the peak fluorescent intensity may correlate with intercalated DNA size (kbp), as that would indicate that the DNA is completely within the interrogation region.

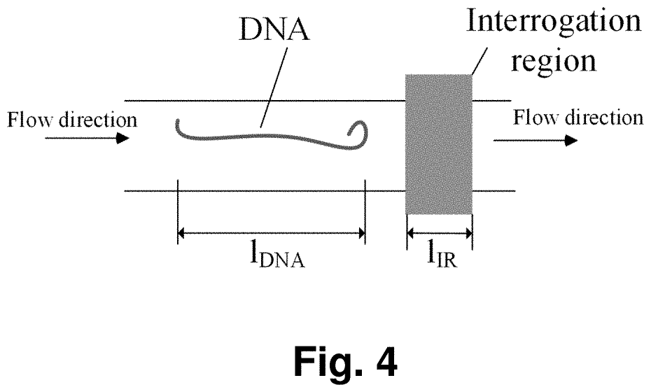

In another case, the physical DNA size 1_{DNA} may be larger than the interrogation region size 1_{IR}, as shown in FIG. 4. This case is more likely when the DNA is in a stretched state. For this case, integrated fluorescent intensity may correlate with intercalated DNA size in kpb.

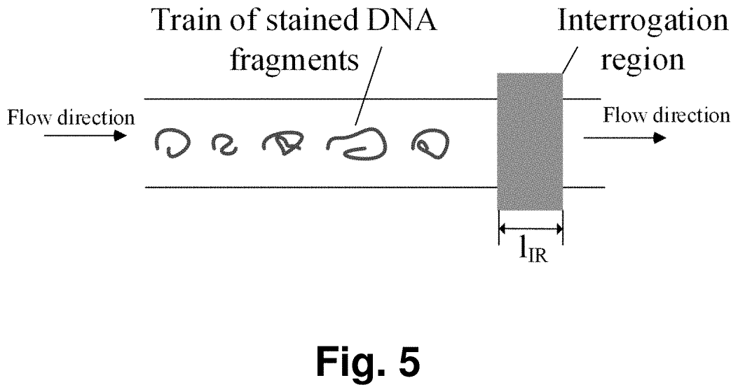

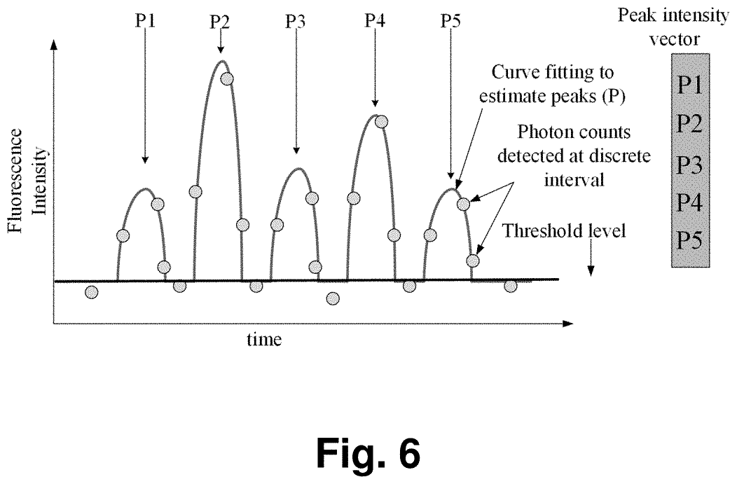

For coiled DNA of size 1μm and below, the case shown in FIG. 3 is the most likely scenario because diffraction limits the minimum interrogation region size to approximately equal to 1 micron for the visible optical platform. In practice, smaller interrogation region size may be preferred to avoid overlapping events. A smaller interrogation region size may also reduce the excitation light intensity requirement. In instances where the excitation illumination has a gaussian intensity or a symmetrical intensity profile, or the DNA physical size is comparable to the interrogation region size, and acquisition of emission fluorescent light is in discrete intervals, then the train of DNA molecule (see FIG. 5) would produce a fluorescence intensity versus time chart as shown FIG. 6. The intensity can then be curve fitted to determine the peak intensity vector.

The peak intensity vector for a single nucleic acid molecule may provide information such as number of RE sites and digested nucleic acid sizes in kbp, which in turn may provide distance between RE sites along the body of molecule. This information allows construction of a nucleic acid restriction map for the original molecule, thus providing a “fingerprint” for the molecule.

A scaling factor ‘s’ to convert the peak intensity vector into a nucleic acid restriction map can be obtained by equating the sum of all the elements in the peak intensity vector and the size of the DNA molecule prior to digestion. The sum of all the elements in the peak intensity vector Pₛᵤₘ obtained from a train of digested DNA fragments originating from a single nucleic molecule entering the device would be given by

Pₛᵤₘ=ΣPᵢ equation (1)

The value Pₛᵤₘ would be proportional to the nucleic acid molecule size entering the fluidic device.

The scaling factor ‘s’ can also be obtained by a calibration procedure. For example, a calibration of an optical platform may involve obtaining fluorescent signal from a standard such as a known intercalated DNA size.

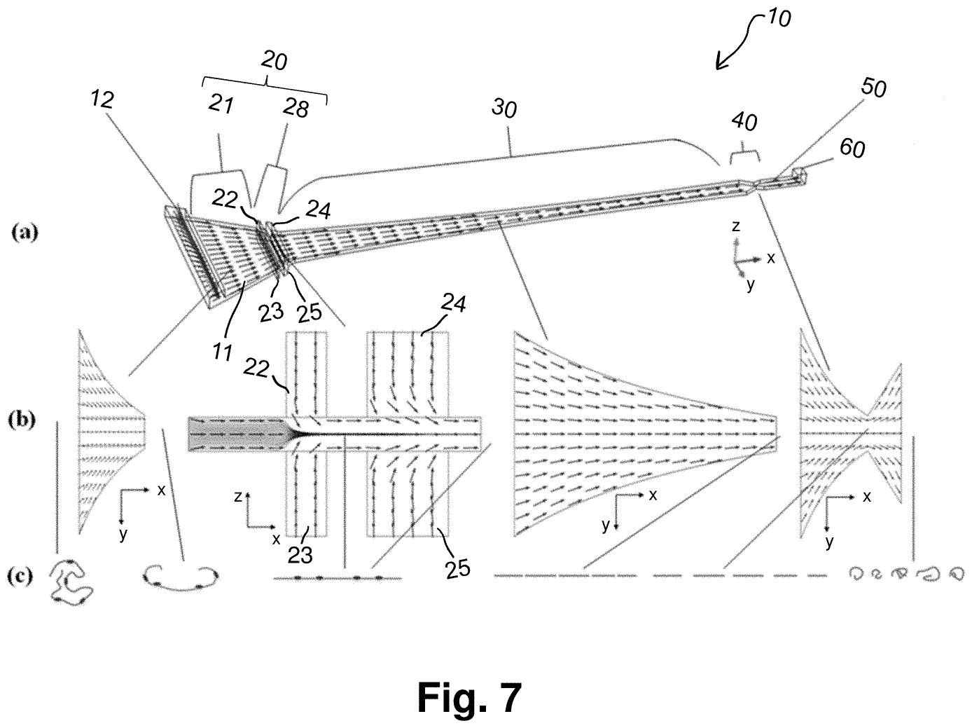

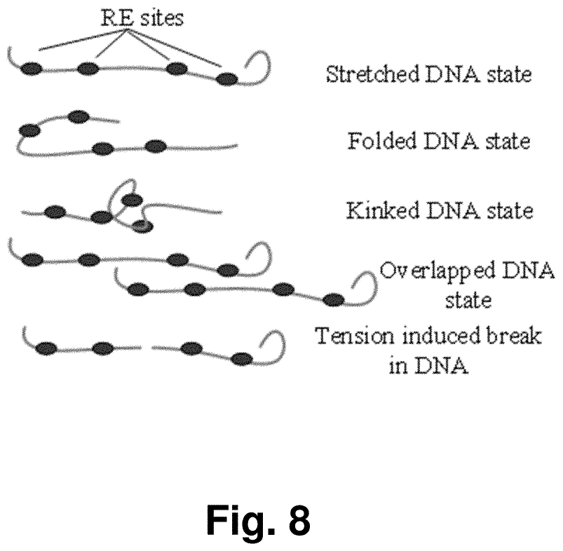

The order of operations to be executed on a single nucleic acid molecule to obtain its fingerprint in the device is shown in FIG. 7. It starts by linearizing/stretching nucleic acid molecules in flow. The stochastic process of the nucleic acid stretching produces various nucleic acid conformational states, as shown in FIG. 8. The stretched DNA state is an ideal state for analysis, while the other states are non-ideal states.

Row (a) of FIG. 7 depicts a perspective view of the device with associated velocity vector fields represented by arrows. Row (b) of the figure shows planar views of the device at various locations of the device. The lines connecting the device of row (a) to the planar views of row (b) indicate the location of each planar view along the device. Row (c) of the figure represents the state of a stained nucleic acid strand at various points along the device as it travels through the device. The embodiment of FIG. 7 includes a fluid flow focusing region 28, which includes supplementary inlets 22, 23, 24 and 25. It should be appreciated that, in the fluid flow focusing region 28, the width of the channel 11 may be converging or may have a constant width. The device may include a pre-stretching zone 21 which may have a converging or a constant width. The pre-stretching zone 21 and fluid flow focusing region 28 may together form the focusing zone 20.

In an ideal scenario, for higher detection efficiency and sensitivity, most of the nucleic acid molecules are well-stretched and non-overlapping before entering the cutting zone of a fluidic device. To achieve a well-stretched and well-separated state, in some embodiments, the nucleic acid molecules pass through an acceleration flow field in a focusing zone of the fluidic device that acts as an acceleration zone and is upstream from the cutting zone, such as the focusing zone 20 shown in FIGS. 2 and 7. The focusing zone may help to create separation between adjacent molecules before their entering the cutting zone. Hydrolysis of the nucleic acid molecules in the cutting zone creates a train of digested fragments. In some embodiments, such as the device shown in FIG. 2, the digested fragments are then flowed through a relaxation zone downstream of the cutting zone. The relaxation zone may help to increase separation between digested fragments.

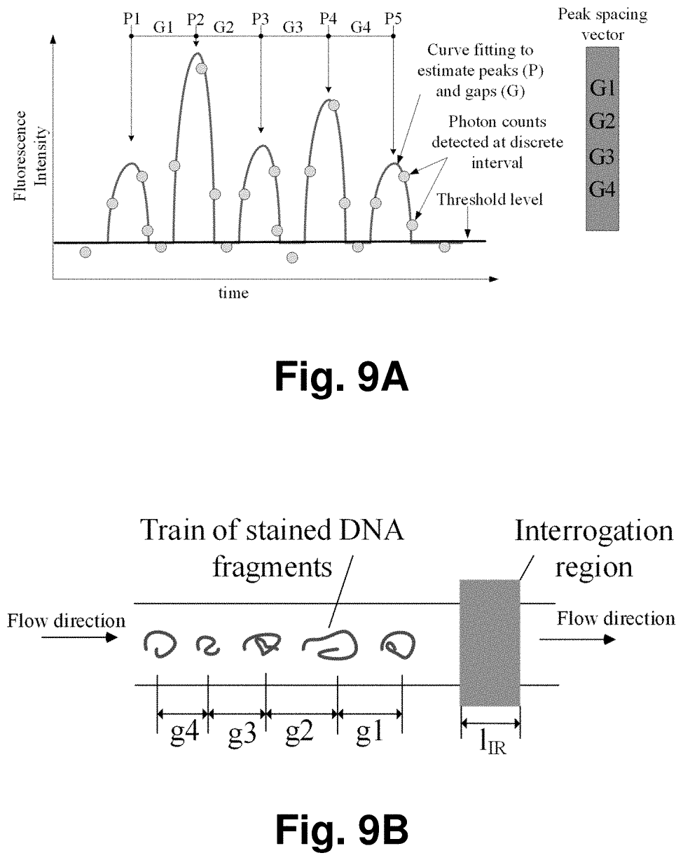

The data of spacing between fluorescence intensity peak signals (FIG. 9A) gives information about spacing between digested fragments (FIG. 9B). Peak spacing vector G in seconds of FIG. 9A, is related to spacing g in meters of FIG. 9B by relationship of G=g/V where V is the average velocity of two fragments with spacing g. This additional information provides a strain field experienced by fragments after digestion. This information can be used to identify and filter the fingerprint from a non-ideal conformation, as discussed below.

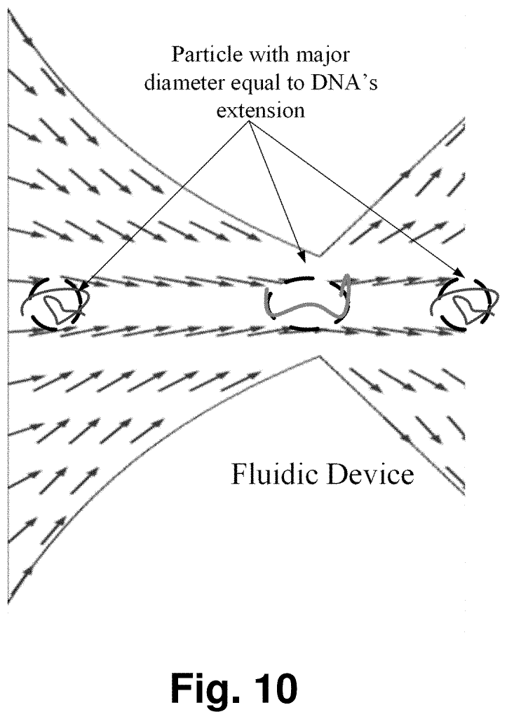

A DNA fragment can be viewed as a soft particle which deforms, translates and rotates in flow. FIG. 10 shows a hypothetical model of a DNA fragment as a particle traveling in flow with its major diameter equal to the fragment's extension. A DNA fragment's extension in flow can be estimated using results of Smith and Chu [ref. 7] and hydrodynamic drag force on non-spherical particles can be evaluated using shape factor corrections [ref. 8].

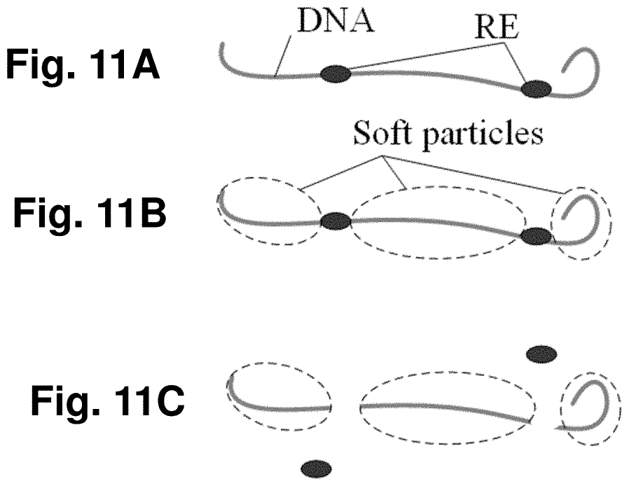

This model can be extended to the DNA-RE complex (see FIGS. 11A-11C), where the complex is seen as chain of particles in close contact at the RE site. On completion of the hydrolysis reaction, the particles can independently move relative to each other. FIG. 11A shows a DNA in a stretched state with REs occupying cognate sites. FIG. 11B shows a hypothetical model representing a DNA-RE complex as a chain of soft particles connected at restriction sites. FIG. 11C shows that, when RE complete hydrolysis, the particles become independent of each other.

When two particles of size d₁ and d₂ that are initially in contact with each other experience a strain rate due to velocity u in direction x, a spacing g, between them develops as given by:

$$g = ( \frac{{d}_{1} + {d}_{2}}{2} ) ( cos α ) exp ( t \cdot \frac{\partial u}{\partial x} ) \qquad equation ( 2 )$$

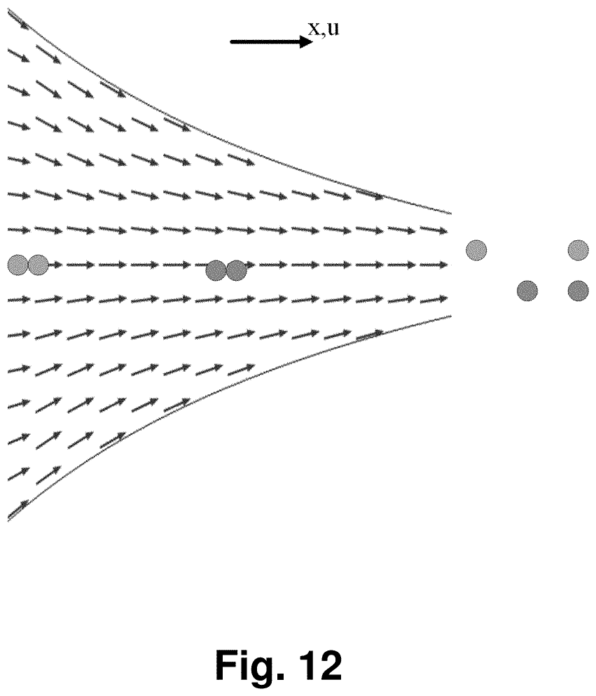

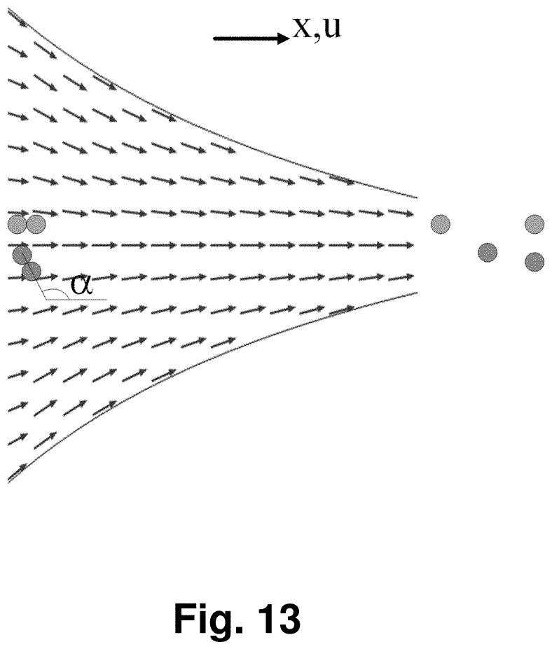

As illustrated in FIG. 13, ‘α’ is the angle enclosed between a line connecting the particle centers and the x axis (axis along direction x). Equation (2) indicates that the spacing g is sensitive to transit time t (see FIG. 12) and the orientation of the particles α at the time of release in flow. FIG. 13 depicts the difference in behavior of two sets of particles released at different orientations in converging flow. The pair of particles having an angle α of zero (e.g. the particles are aligned with the x-axis) exhibited greater spacing than the particles having a non-zero angle α.

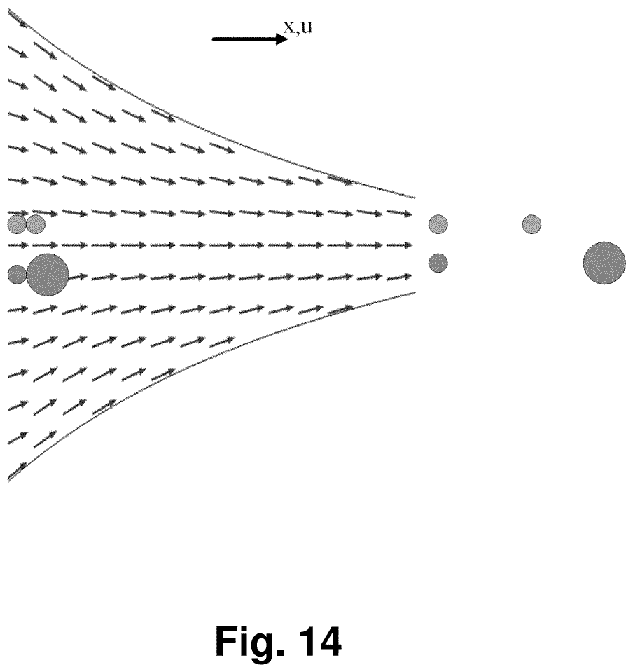

In addition, spacing g is related to particle size d. Larger particles will have higher spacing. FIG. 14 depicts the difference in behavior of two sets of particles: the first pair having equal size d1 and the second pair having unequal size d1 and d2, where d2 is greater than d1.

In equation (2), the term within the exponential can be referred to as accumulated strain, ε given by

$$e = ( t \cdot \frac{\partial u}{\partial x} ) \qquad equation ( 3 )$$

For a particle pair in an accelerating flow field, velocity u increases as the particles travel in flow, increasing the amount of spacing between nearby particles. Also, in some embodiments, the flow fields in the fluidic device shown in FIG. 7 are such that all the different types of zones, such as the focusing zone, the cutting zone and the relaxation zone, increase overall fluid velocity and hence lead to increase in particle spacing.

To compare gaps between particles of different size for purpose of extracting information about accumulated strain and orientation, equation (2) can be rewritten as:

$$\frac{2 {g}_{1}}{{d}_{1} + {d}_{2}} = ( cos α ) exp ( t \cdot \frac{\partial u}{\partial x} ) \qquad equation ( 4 )$$

The left-hand side term in equation (4) can be called a normalized gap, gₙₒᵣₘ, written as:

$${g}_{norm , 1} = \frac{2 {g}_{1}}{{d}_{1} + {d}_{2}} \qquad equation ( 5 )$$

Equations (4) and (5) can be generalized to include spacing between other nearby fragments in the nucleic acid chain. The significance of gₙₒᵣₘ is that the normalized gap is independent of particle size. For example, the case of unequal particle size discussed previously (see FIG. 14) would show larger spacing for large particles, but normalized gap gₙₒᵣₘ would be equal as the accumulated strain and orientation is the same for the two particle pairs.

When estimating gₙₒᵣₘ using equation (5) for a train of digested nucleic acid fragments, gₙₒᵣₘ can be estimated using the peak intensity vector and the peak spacing vector.

For well-stretched molecules undergoing hydrolysis by a RE in a steady state flow field as shown in FIG. 7, the right hand side of equation (4) is expected to be deterministic. A steady state flow field is described as one which is stationary and/or does not change with time at any position in a fluidic device.

In an ideal scenario, molecules are well-stretched and well-separated from other adjacent molecules before entry into the cutting zone. In some embodiments, this may be accomplished by flowing the molecules through an acceleration flow field in a focusing zone prior to entry into the cutting zone.

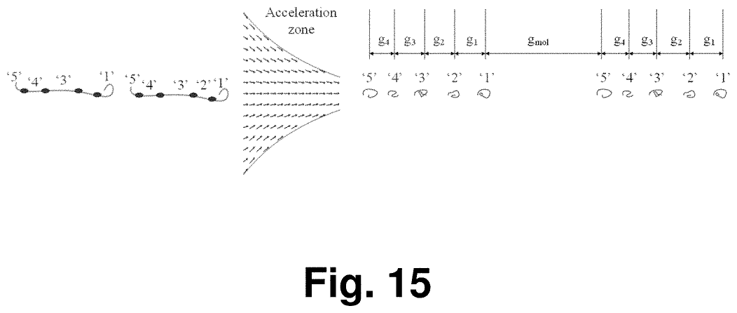

In some embodiments, the cutting zone and the relaxation zone are shaped to give rise to an accumulated strain in the digested fragments, helping to form gaps between the digested fragments, where the gaps are labeled as g₁, g₂, g₃, and g₄ in FIG. 15. The gap between successive fragments from different molecules, labeled as “gₘₒₗ” in FIG. 15, will be much larger than gaps between successive fragments from the same original nucleic acid molecule. This difference helps to determine whether a gap represents spacing between two different molecules or spacing between two fragments within the same molecule. Another approach would be to use the normalized gap gₙₒᵣₘ from equation (5). Natural variations in hydrolysis rates and stretching dynamics for each individual nucleic acid would lead to gₙₒᵣₘ having an acceptable range with a lower limit and an upper limit. A gₙₒᵣₘ greater than a lower limit of threshold value, g_{thres,low}, indicates that there is proper separation of digested molecules from each other.

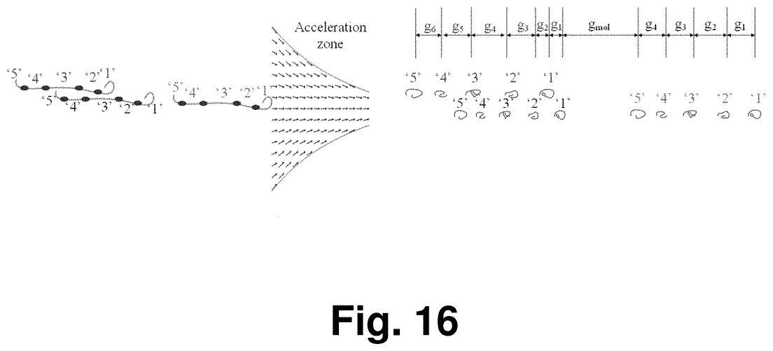

In some instances, the nucleic acid molecules may enter the cutting zone in an overlapped state, where one molecule overlaps with another, as shown in FIG. 16. In this case, the normalized gap gₙₒᵣₘ between some of these fragments would be smaller than the lower threshold value g_{thres,low}, indicating that a non-ideal conformation has occurred. Also, for overlapped molecules, the sum of all the elements Pₛᵤₘ in the peak intensity vector from equation (1) would be higher than expected. A higher than expected Pₛᵤₘ would indicate that the molecule was likely in an overlapped state during cutting, and thus the measurements associated with this molecule should be filtered out.

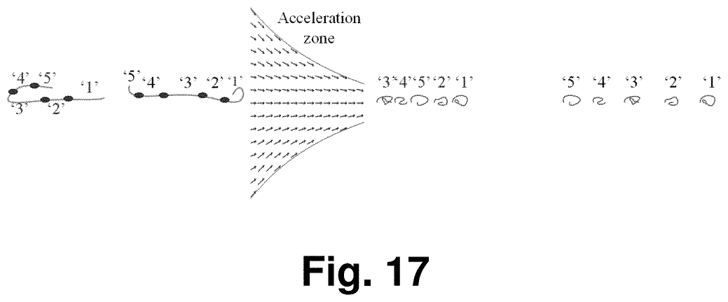

In some instances, a nucleic acid molecule can enter the cutting zone in a folded state, such as in FIG. 17. In this case, similar to the overlapped state, the normalized gap gₙₒᵣₘ between these fragments would be smaller than the lower threshold value, g_{thres,low}.

In some instances, a nucleic acid molecule can enter the cutting zone in a kinked state, as illustrated in FIG. 8 with the label “kinked DNA state.” Like the folded state and the overlapped state, the kinked state could lead to RE sites on nucleic acids that have not been linearized. That case would produce a normalized gap gₙₒᵣₘ that is smaller than the lower threshold value, g_{thres,low}.

In some instances, a nucleic acid molecule can break in the focusing zone, e.g. in an area of high or highest strain rate. As illustrated in FIG. 8 with the label “tension induced break in DNA,” such a situation would lead to two halves seeing accumulated strain from the focusing zone, cutting zone and the relaxation zone. The two halves would generate two different trains of digested fragments. The sum of all the elements Pₛᵤₘ of the peak intensity vector would be smaller than expected, indicating the presence of a broken molecule that should be filtered out.

The above cases show that thresholds for gₙₒᵣₘ and Pₛᵤₘ can be used to indicate whether molecules are in an ideal state or non-ideal state when entering the cutting zone of the fluidic devices.

In some approaches, estimation of limits may be used to determine the thresholds for gₙₒᵣₘ and Pₛᵤₘ. The estimation of limits can be performed using computational fluid dynamics (CFD) packages such as COMSOL Multiphysics or using statistical methods on experimental data. Two approaches for estimating the thresholds for gₙₒᵣₘ and Pₛᵤₘ are discussed below.

In some embodiments, estimation of the thresholds is primarily performed using CFD and a scaling factor ‘s’. As shown in equation (4) and (5), the normalized gap gₙₒᵣₘ is a function of accumulated strain. Using computational fluid dynamics (CFD), the strain rate field, velocity field and species concentration field in the fluidic device can be obtained. Hydrolysis reaction rates for REs, such as BamhI, SmaI and EcorV, have been measured to be around 0.2 s-1 [refs. 9,10]. This information can be used to estimate the time and location along the fluidic device where digestion would be completed. Then, the right-hand term in equation (4) can be estimated. Further, on selection of appropriate tolerances of the threshold values of gₙₒᵣₘ, the lower and upper thresholds can be determined. To determine the appropriate or best guess thresholds for gₙₒᵣₘ, the variation measured and reported in hydrolysis reaction rates, can be used, such as in [ref. 9]: van den Broek et al., DNA-tension dependence of restriction enzyme activity reveals mechanochemical properties of the reaction pathway. Nucleic acids research, 33(8), pp.2676-2684.

In determining the thresholds for Pₛᵤₘ, the scaling factor ‘s’ and the size distribution of the input nucleic acid molecules entering the fluidic device would give the expected value of Pₛᵤₘ and its distribution.

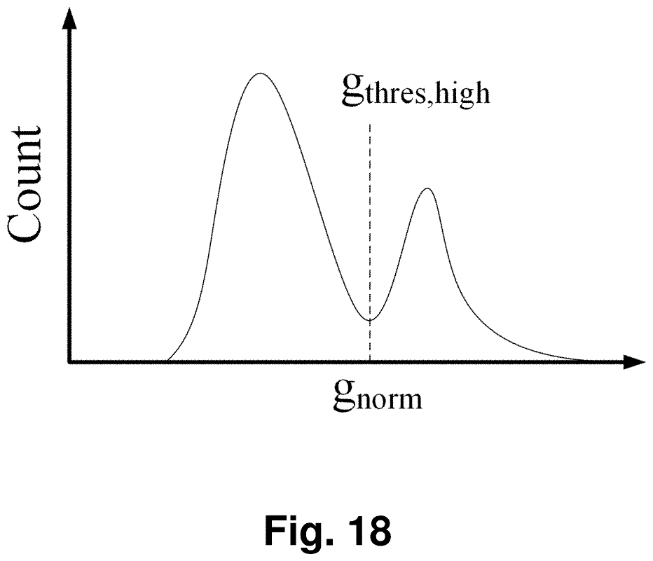

In other embodiments, estimation of the thresholds is performed by using the data collected from a run where trains of digested nucleic acid fragments are generated from nucleic acid molecules entering the cutting zone of the device. Peak spacing data and peak intensity data from such a fluidic device can be plotted as a histogram of normalized gap gₙₒᵣₘ, e.g. looking similar to what is shown in FIG. 18. The large peak at lower values of gₙₒᵣₘ (left peak in FIG. 18) represents spacing between digested fragments and is related to the hydrolysis time of RE. The smaller peak at higher values of gₙₒᵣₘ (right peak in FIG. 18) represents spacing gₘₒₗ between adjacent fragments on distinct but nearby trains. gₘₒₗ is related to concentration of nucleic acid molecules entering the fluidic device. An upper threshold g_{thres,high} can then be appropriately chosen to distinguish data representing spacing between trains of digested fragments within the same molecule from data representing spacing between nucleic acid molecules. If the two peaks are distinct and non-overlapping (i.e. bins between the peaks have counts or frequency going to near zero) then an appropriate way to choose g_{thres,high} would be to use an upper limit based on the standard deviation estimated from values in the left peak only. Another way would be to use a percentile-based limit, e.g., the 90^{th} percentile can be used for g_{thres,high}. If the two peaks overlap, such as shown in FIG. 18, a local minimum between two peaks could be used for g_{thres,high}.

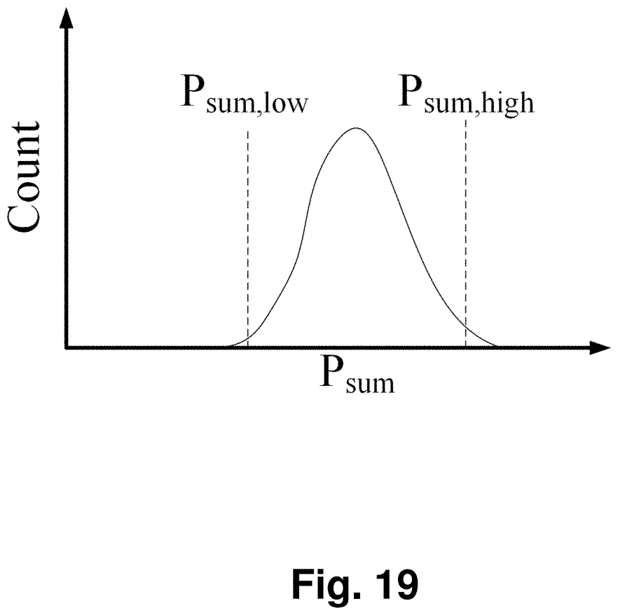

Once the trains of digested fragments from within the same nucleic acid molecule are identified (left peak in FIG. 18), the peak intensity vector for each such isolated train can be obtained. This would allow estimating the sum of elements in the peak intensity vector for each isolated train, Pₛᵤₘ. Plotting a histogram of Pₛᵤₘ would allow setting of an upper threshold P_{sum,high} and a lower threshold P_{sum,low} to filter out the trains of fragments coming from nucleic acid molecules which are overlapped or broken due to tension. One example of such a histogram is shown in FIG. 19. The thresholds can be determined via statistical analysis. For example, standard deviation based-limits may be used, e.g. first finding the standard deviation and average value for Pₛᵤₘ, then setting the upper limit and lower limit to an over and under average value by two to three standard deviations. Another example of finding thresholds using statistical analysis would be to use limits based on percentile, e.g. where the lower limit is set to the 10^{th} percentile and the upper limit is set to the 90^{th} percentile. In the case of known input nucleic acids size range, thresholds can be set after converting DNA size range into expected variations in Pₛᵤₘ using scaling factor ‘s’. The size range of input nucleic acids can be estimated using gel electrophoresis, or by running intercalated DNA, without an RE digestion step, through the detection region of the fluidic device. Another approach to determine upper and lower thresholds for Pₛᵤₘ involves taking user input for DNA size range to be used for further analysis. The DNA size range can be converted into threshold limits, P_{sum,high} and P_{sum,low}, using scaling factor ‘s’. The basis for the lower limit for DNA size selection can be based on bioinformatics as shorter DNA sizes are expected to give less information to make identification of bacterial genome. The upper limit for DNA size selection can be based on nucleic acid dynamics in the fluidic device as DNA that are longer than the device was designed for are expected to stretch incompletely or poorly.

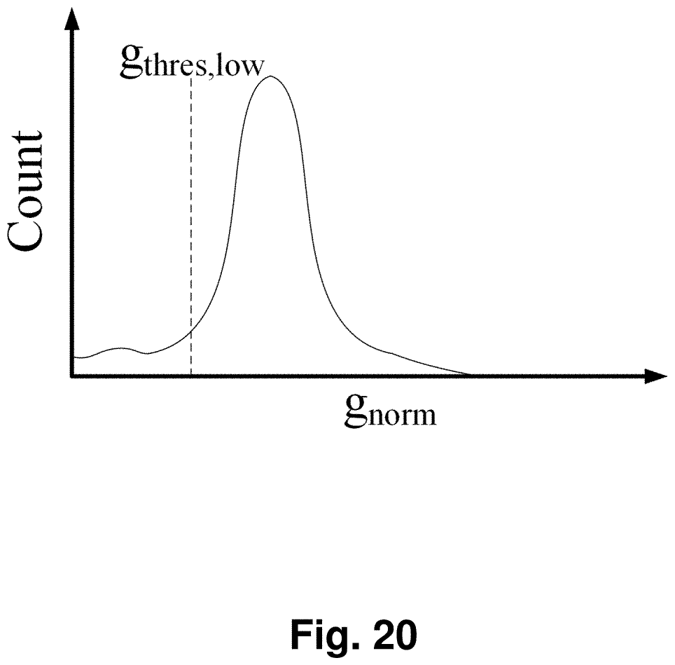

The normalized gap gₙₒᵣₘ between digested fragments from within the same nucleic acid molecule (left peak in FIG. 18) can also be plotted to determine the lower threshold value, g_{thres,low}. An example is shown in FIG. 20. As with the Pₛᵤₘ thresholds, this threshold can be determined via statistical analysis, e.g. 2 or 3-sigma limits below the mean of gₙₒᵣₘ's distribution or the 10^{th} percentile limit of gₙₒᵣₘ's distribution. Another way to obtain g_{thres,low} is to use a reference nucleic acid with a known sequence, such as lambda DNA. In that case, the measured signature or DNA map can be compared to an expected one to obtain an appropriate threshold value for g_{thres,low}. This lower threshold can be used to filter out folded molecules, kinked molecules and overlapped molecules.

As an illustrative embodiment, an example of an algorithm is as follows:
a) Use discrete fluorescent intensity signal collected in a detection channel, and a threshold for background signal, to identify signal from stained digested nucleic acid fragments.
b) For each fragment successively numbered ‘i’, estimate peak signal Pᵢ (in photon counts) and spacing, Gᵢ (in seconds) between successive signal peaks Pᵢ and Pᵢ₊₁.
c) Estimate the normalized gap, g_{norm,i}, using equation (6) below:

$${g}_{norm , i} = ( \frac{2 v}{s} ) ( \frac{{G}_{i}}{{P}_{i} + {P}_{i + 1}} ) \qquad equation ( 6 )$$

Here, ‘v’ is average velocity of the nucleic acid fragments and ‘s’ is the scaling factor to convert fluorescent signal to the nucleic acid size. ‘v’ and ‘s’ are expected to be constant and dependent on the fluidic device and the optical detection system only.
d) Plot a histogram of gₙₒᵣₘ and choose an upper threshold g_{thres,high} as illustrated in FIG. 18.
e) Using conditional statement (7) below, identify normalized gaps, g_{norm,i}, between two neighboring digested fragments associated with two different parent nucleic acid molecules entering the fluidic device:

g_{norm,i}≥g_{thres,high} equation (7)
f) Convert the one-dimensional vectors, Pᵢ and g_{norm,i}, into two dimensional matrices, P_{m,n} and g_{norm,m,n}. Each row ‘m’ is associated with a train of digested nucleic acid fragments coming from the nucleic acid molecule ‘n’ entering the fluidic device. If conditional statement (7) is true, then the g_{norm,i+1} and P₁₊₁ from the subsequent digested fragment ‘i+1’ is associated with the next parent nucleic acid molecule ‘n+1’, as represented by equation (8a).

g_{norm,1,n+1}=g_{norm,i+1}

P_{1,n+1}=Pᵢ₊₁ equation (8a)
g) If conditional statement (7) is false, then the g_{norm,i+1} and Pᵢ₊₁ from the subsequent digested fragment ‘i+1’ is associated with same parent nucleic acid molecule ‘n’, as represented by equation (8b).

g_{norm,m+1,n}=g_{norm,i+1}

P_{m+1,n}=Pᵢ₊₁ equation (8b)
h) Compute P_{sum,n} using equation (9) below and plot it on a histogram to choose lower and upper thresholds for Pₛᵤₘ, as illustrated in FIG. 19.

$${P}_{sum , n} = {\sum }_{i} {P}_{i , n} \qquad equation ( 9 )$$

i) Use P_{sum,low} and P_{sum,high} and either conditional statement from equation (10) below to find and eliminate columns associated with overlapped and tension damaged nucleic acid molecules. Then rearrange the matrices P_{m,n} and g_{norm,m,n}.

P_{sum,i}≥P_{sum,low}

P_{sum,i}≥P_{sum,high} equation (10)
j) Plot a histogram of all the elements in matrix, g_{norm,m,n}, and choose g_{thres,low}, as illustrated in FIG. 20.
k) Use g_{thres,low} along with the conditional statement in equation (11) below to identify digested fragments associated with nucleic acid molecule ‘n’ entering the cutting zone of a fluidic device in a folded or kinked or overlapped state. Eliminate the columns and rearrange the matrix, P_{m,n}.

g_{norm,i,n}≥g_{thres,low} equation (11)
l) Use P_{m,n} with scaling factor, ‘s’, to construct the RE map for nucleic acid molecule ‘n’.

### REFERENCES

1. Goodwin, P. M., Johnson, M. E., Martin, J. C., Ambrose, W. P., Marrone, B. L., Jett, J. H. and Keller, R. A., 1993. Rapid sizing of individual fluorescently stained DNA fragments by flow cytometry. Nucleic Acids Research, 21(4), pp.803-806.

2. Van Orden, A., Keller, R. A. and Ambrose, W. P., 2000. High-throughput flow cytometric DNA fragment sizing. Analytical chemistry, 72(1), pp.37-41.

3. Habbersett, R. C. and Jett, J. H., 2004. An analytical system based on a compact flow cytometer for DNA fragment sizing and single-molecule detection. Cytometry Part A, 60(2), pp.125-134.

4. Chan, E. Y., Goncalves, N. M., Haeusler, R. A., Hatch, A. J., Larson, J. W., Maletta, A. M., Yantz, G. R., Carstea, E. D., Fuchs, M., Wong, G. G. and Gullans, S. R., 2004. DNA mapping using microfluidic stretching and single-molecule detection of fluorescent site-specific tags. Genome research, 14(6), pp.1137-1146.

5. Wong, P. K., Lee, Y. K. and Ho, C. M., 2003. Deformation of DNA molecules by hydrodynamic focusing. Journal of Fluid Mechanics, 497, pp.55-65.

6. Larson, J. W., Yantz, G. R., Zhong, Q., Charnas, R., D'Antoni, C. M., Gallo, M. V., Gillis, K. A., Neely, L. A., Phillips, K. M., Wong, G. G. and Gullans, S. R., 2006. Single DNA molecule stretching in sudden mixed shear and elongational microflows. Lab on a Chip, 6(9), pp.1187-1199.

7. Smith, D. E. and Chu, S., 1998. Response of flexible polymers to a sudden elongational flow. Science, 281(5381), pp.1335-1340.

8. Hidy, G., 2012. Aerosols: an industrial and environmental science. Elsevier.

9. van den Broek, B., Noom, M. C. and Wuite, G. J., 2005. DNA-tension dependence of restriction enzyme activity reveals mechanochemical properties of the reaction pathway. Nucleic acids research, 33(8), pp.2676-2684.

10. Riehn, R., Lu, M., Wang, Y. M., Lim, S. F., Cox, E. C. and Austin, R. H., 2005. Restriction mapping in nanofluidic devices. Proceedings of the National Academy of Sciences of the United States of America, 102(29), pp.10012-10016.

1. A method in relation to identifying a nucleic acid of a sample, the method being for determining whether a molecule of the sample exhibits a predetermined physical characteristic, the method comprising acts of:
(A) determining a distance g between a first fragment and a second fragment of the sample, based at least in part upon an amount of time G between a first signal vector element P1 representing emission of light by the first fragment and a second signal vector element P2 representing emission of light by the second fragment, the first signal vector element P1 and second signal vector element P2 contributing to a sum Psum of all elements of signal vectors representing emission of light by a plurality of fragments including the first fragment and the second fragment;
(B) determining at least one of a distance threshold and a signal vector threshold;
(C) performing a comparison between the distance and the distance threshold, between the sum of all the elements of signal vectors and the signal vector threshold, or between the distance and the distance threshold and between the sum of all the elements of signal vectors and the signal vector threshold; and
(D) determining based at least in part upon the comparison performed in the act (C) whether the molecule exhibits the predetermined physical characteristic.

2. The method of claim 1, wherein the nucleic acid is DNA.

3. The method of claim 1, wherein the first and second fragments have respective first and second size in base pairs, and the distance g determined in the act (A) is a normalized distance gₙₒᵣₘ, the distance gₙₒᵣₘbeing normalized to account for the first and second size in base pairs of the first and second fragments.

4. The method of claim 3, wherein gₙₒᵣₘ is determined using an equation:
$${g}_{norm} = ( \frac{2 v}{s} ) ( \frac{G}{P 1 + P 2} )$$
where v is average velocity of the first fragment and the second fragment and s is a scaling factor to convert emission of light to fragment size.

5. The method of claim 4, wherein the act (B) comprises determining an upper distance threshold g_{thres,high} by plotting a histogram of gₙₒᵣₘ, the histogram having a left peak and a right peak that are distinct and non-overlapping, and setting the upper distance threshold g_{thres,high} to be two standard deviations above a mean of values in the left peak.

6. The method of claim 4, wherein the act (B) comprises determining an upper distance threshold g_{thres,high} by plotting a histogram of gₙₒᵣₘ, the histogram having a left peak and a right peak that are distinct and non-overlapping, and setting the upper distance threshold g_{thres,high} to be three standard deviations above a mean of values in the left peak.

7. The method of claim 4, wherein the act (B) comprises determining an upper distance threshold g_{thres,high} by plotting a histogram of gₙₒᵣₘ, the histogram having a left peak and a right peak that are distinct and non-overlapping, and setting the upper distance threshold g_{thres,high} to be at a 90^{th} percentile of values of the left peak.

8. The method of claim 4, wherein the act (B) comprises determining an upper distance threshold g_{thres,high} by plotting a histogram of gₙₒᵣₘ, the histogram having a left peak and a right peak that are overlapping, and setting the upper distance threshold g_{thres,high} to be a local minimum of values between the left peak and the right peak.

9. The method of claim 5, further comprising using the upper distance threshold g_{thres,high} to associate digested fragments with their parent nucleic acid using a conditional statement:
g_{norm,i}≥g_{thres,high}
where g_{norm,i} is normalized gaps between two neighboring digested fragments associated with two different parent nucleic acid molecules entering a fluid device;
if the conditional statement is true, then the g_{norm,i+1} and Pᵢ₊₁ from a subsequent digested fragment ‘i+1’ is associated with a next parent nucleic acid molecule ‘n+1’; and
if the conditional statement is false, then the g_{norm,i+1} and Pᵢ₊₁ from the subsequent digested fragment ‘i+1’ is associated with a same parent nucleic acid molecule ‘n’.

10. The method of claim 1, wherein the act (B) comprises determining a lower signal vector threshold P_{sum,low} by setting P_{sum,low} to be two standard deviations below a mean of values of Psum.

11. The method of claim 1, wherein the act (B) comprises determining a lower signal vector threshold P_{sum,low} by setting P_{sum,low} to be three standard deviations below a mean of values of Psum.

12. The method of claim 1, wherein the act (B) comprises determining a lower signal vector threshold P_{sum,low} by setting P_{sum,low} to be at a 10th percentile of values of Psum.

13. The method of claim 1, wherein the act (B) comprises determining an upper signal vector threshold P_{sum,high} by setting P_{sum,high} to be two standard deviations above a mean of values of Psum.

14. The method of claim 1, wherein the act (B) comprises determining an upper signal vector threshold P_{sum,high} by setting P_{sum,high} to be three standard deviations above a mean of values of Psum.

15. The method of claim 1, wherein the act (B) comprises determining an upper signal vector threshold P_{sum,high} by setting P_{sum,high} to be at a 90th percentile of values of Psum.

16. The method of claim 1, wherein the predetermined physical characteristic comprises the molecule being overlapped with a second molecule, and wherein the act (D) comprises determining whether the molecule is overlapped with the second molecule based at least in part upon a comparison between the distance and the distance threshold.

17. The method of claim 16, wherein the act (D) comprises determining whether the molecule is overlapped with the second molecule based at least in part upon the distance being less than the distance threshold.

18. The method of claim 1, wherein the predetermined physical characteristic comprises the molecule being overlapped with a second molecule, and wherein the act (D) comprises determining whether the molecule is overlapped with the second molecule based at least in part upon a comparison between the sum of all the elements of signal vectors and the signal vector threshold.

19. The method of claim 18, wherein the act (D) comprises determining whether the molecule is overlapped with the second molecule based at least in part upon the sum of all the elements of signal vectors exceeding the signal vector threshold.

20. The method of claim 1, wherein the predetermined physical characteristic comprises the molecule being folded, and wherein the act (D) comprises determining whether the molecule is folded based at least in part upon a comparison between the distance and the distance threshold.

21. The method of claim 20, wherein the act (D) comprises determining whether the molecule is folded based at least in part upon the distance being less than the distance threshold.

22. The method of claim 1, wherein the predetermined physical characteristic comprises the molecule being kinked, and wherein the act (D) comprises determining whether the molecule is kinked based at least in part upon a comparison between the distance and the distance threshold.

23. The method of claim 22, wherein the act (D) comprises determining whether the molecule is kinked based at least in part upon the distance being less than the distance threshold.

24. The method of claim 1, wherein the predetermined physical characteristic comprises the molecule having a tension-induced break, and wherein the act (D) comprises determining whether the molecule has a tension-induced break based at least in part upon a comparison between the sum of all the elements of signal vectors and the signal vector threshold.

25. The method of claim 24, wherein the act (D) comprises determining whether the molecule has a tension-induced break based at least in part upon the sum of all the elements of signal vectors being less than the signal vector threshold.
